(19) United States
(12) Patent Application Publication
(10) Pub. No.: US 2003/0229392 A1
(43) Pub. Date: Dec. 11, 2003

(54) Drug eluted vascular graft

(76) Inventor: Samuel J. Wong, Orinda, CA (US)

Correspondence Address: SAMUEL J. WONG, 3 La Cuesta Road, Orinda, CA, 94563, US

(21) Appl. No.: 10/443,722

(22) Filed: May 23, 2003

Related U.S. Application Data
(60) Provisional application No. 60/384,677, filed on Jun. 3, 2002.

(51) Int. Cl. A61F 2/06

(52) U.S. Cl. 623/1.42; 623/1.43; 623/1.46

(57) ABSTRACT

A drug Eluted vascular graft where the internal lumen are coated with at least one or more bioerodible polymers capable of releasing at least one or more therapeutic agents in a controlled time-released manner. The therapeutic agent may include an antimicrotubule, an antiproliferative agent, or an antithrombogenic agent.

A method for preventing thrombosis of vascular grafts used in vascular access for hemodialysis, vascular reconstruction and coronary bypass grafting.

This application claims the benefit of the provisional application 60/384,677 filed Jun. 3, 2002

### BACKGROUND OF THE INVENTION

1. Field of the Invention

The invention relates to vascular grafts which are used in medical practice as an artificial conduit for blood, wherein the Drug Eluted Vascular Graft has at least one bioerodible polymer capable of releasing at least one therapeutic agent in a controlled and sustained manner over a prolonged period of time as a method to prevent restenosis and thrombosis.

2. Background of the Art

Vascular grafts are medical devices used as an artificial conduit for bodily fluids, usually blood. When used for hemodialysis the vascular graft serves as a nonstatic reservoir of blood, where blood is readily accessible to a dialysis machine. The vascular graft serves as a life line, an essential interface between the patient and the dialysis machine.

When used in the treatment of peripheral vascular disease and coronary artery disease, vascular grafts serve as an artificial conduit for blood bypassing diseased blood vessels and thus supplying blood to the ischemic organ. In coronary artery bypass grafting, artificial vascular grafts are rarely used due to their high incidence of thrombosis. The current graft material of choice is to use a native blood vessel such as the left internal mammary artery or saphenous vein. Unfortunately these also have significant thrombosis rates, although substantially less than artificial grafts.

Thrombosis of vascular grafts is problematic. The most common cause of thrombosis is stenosis within the internal lumen of the vascular graft. Stenosis occurs as a result of the body's natural healing mechanism. When a vascular graft is implanted, injury occurs to the arterial or venous system to which the vascular graft is sutured to. The vascular graft itself is a foreign body. Thru a complex process smooth muscle cells migrate onto the internal lumen. As these smooth muscle cells proliferate, they form a neointima known in the art as neointimal hyperplasia. Over time the neointimal hyperplasia progresses, causing a reduction in an internal diameter of the internal lumen and eventually thrombosis ensues.

Subsequently, there have been many attempts to render the grafts less thrombogenic. Anticoagulants such as heparin in a polymer coating and non-thrombogenic polymers have been tried with little success. Smooth muscle cell proliferation with neointimal hyperplasia develop and restenosis and thrombosis inevitably ensues.

Various therapeutic agents have been demonstrated to prevent or retard neoimtimal hyperplasia. Antimicrotuble agents such as paclictaxel and docetaxel inhibit mitosis and hence cellular proliferation. Antiproliferative agents such as cyclophosphamide, mithromycin, and actinomycin-D prevent proliferation of smooth muscle cells. Sirolimus, cyclosporine A, dexamethasone and methyl prednisolone are immunosuppressive agents that have been also shown to prevent or retard neointimal hyperplasia.

Recently, drug eluted stents containing an antiproliferative agents such as paclictaxel and sirolimus has been demonstrated to at least-retard if not prevent restenosis within the stent. (U.S. Pat. No. 6,379,382 Yang and U.S. Pat. No. 6,273,913 Wright et al.)

Accordingly, it would be desirable to construct a vascular graft capable of delivering a therapeutic agent with similar antiproliferative properties to prevent restenosis. Ideally, the therapeutic agent would be released locally in a continuous and sustained manner.

### SUMMARY OF THE INVENTION

The purpose of the invention is to provide an improved vascular graft capable of preventing or retarding thrombosis within said vascular graft. The graft may be constructed with preexisting material commonly used such as ePTFE but would include the use of at least one bioerodible polymer which would release a least one therapeutic agent with antiproliferative or antithrombogenic properties in a controlled and sustained manner over a prolonged period of time.

The release rate of therapeutic agents from the bioerodible polymer is controlled-by variations in the structure and formulation of the polymer, the solvent, and the ratio of polymer to drug.

In addition, a coating substance may be applied to the therapeutic agent to provide biocompatibility or to control drug release.

### DETAILED DESCRIPTION OF THE INVENTION

The primary embodiment of the invention is to provide an Eluted vascular graft that when used for hemodialysis is capable of preventing neointimal hyperplasia and subsequent thrombosis of said vascular graft. The Eluted vascular graft is implanted underneath the skin with one end sutured to an artery and the other sutured to a vein. Blood is then diverted from the artery into the drug Eluted vascular graft and then empties in the vein. The internal lumen of the vascular graft is composed of at least one layer containing least one bioerodible polymer that would continuously release at least one anti-stenotic alone or may include and or anti thrombotic agent. An example may be paclictaxel or sirolimus only or paclictaxel and sirolimus, or sirolimus and dexamethasone, or sirolimus and dexamethasone and clopidogrel.

A second embodiment, the invention is used as a vascular reconstruction in peripheral vascular disease. The drug Eluted vascular graft is implanted inside the affected organ with one end of the vascular graft sutured proximally to the diseased blood vessel (usually an artery) and the other attached distal to the diseased blood vessel. Again the internal lumen would be composed of at least one bioerodible polymer and continuously release at least one therapeutic agent as described in the first embodiment.

A third embodiment, the invention is used in coronary artery bypass grafting. The drug Eluted vascular graft is used as an artificial conduit diverting blood around the diseased artery. The size is usually of smaller caliber: 4 mm or less. As with the first embodiment at least one therapeutic agent is interspersed within the bioerodible polymer.

The shell of the drug eluted vascular graft may be constructed with ePTFE, polyvinylchloride polypropylene, florinated ethylene propylene, polyetherurethaneura or other biocompatible plastics. The inner coat would consist of at least one layer of bioerodible polymer such as but not limited to the bioerodible polymers described in U.S. Pat. No. 4,131,648 Choi et al., U.S. Pat. No. 4,282,201, Choi. In addition a erosion rate modifier may be included to aid in regulating the rate of erosion of the polymer over time as described in U.S. Pat. No. 4,346,709 Schmitt.

Embedded within the bioerodible polymer would be at least one therapeutic agent. An antiproliferative agent such as sirolimus and paclictaxel might be advantageous given the initial success in stents however other antiproliferative agents such as cyclophosphamide, actinomycin D, mitomycin, steroid, angiotensin inhibitor, nitric oxide donor, calcium channel blocker, anti-sense nucleic acid, thiazolidinedione, or HMG Co A reductase inhibitor may afford similar results as shown in animal models.

The internal lumen of the vascular graft would be composed of at least one erodible polymer and at least one therapeutic agent. The internal lumen may comprised of two or more layers of polymer(s) with the therapeutic agent sandwiched in between each layer (FIG. 2) or different layers of a mixture of polymer/therapeutic agent(s)(FIG. 3).

The polymer, therapeutic agent or mixture of polymer/therapeutic agent may be applied to the vascular graft using conventional dip-coating or spray coating techniques where the polymer and therapeutic agent may be suspended in an organic solvent. The solvent then evaporates leaving a coat/layer of polymer or therapeutic agent.

Coating may also be achieved by vapor deposition, plasma polymerization or using an air suspension process as described in U.S. Pat. No. 6,368,658 Schwarz et al.

Alternatively, layers of polymer and therapeutic agents can be achieved using electron beam deposition, electron beam polymerization, or electron beam treatment process.

### DESCRIPTION OF DRAWINGS

FIG. 1A. Longitudinal and end view of the invention where L is the length of the vascular graft and d1 and d2 are the diameter of the vascular graft. The length the vascular graft is variable depending on the desired length and d1 and d2 may be variable, usually 4 to 8 mm in diameter.

FIG. 1B. Three dimension view of the invention where L and d1 and d2 are as described above.

FIG. 2. Cross section of invention illustrating vascular graft in hatched lines and illustration of a therapeutic agent(s) embedded within one or more layer of bioerodible polymer.

FIG. 3. Cross section of invention illustrating vascular graft in hatched lines and polymer/therapeutic agent layer.

FIG. 4. Illustration of drug eluted vascular graft for vascular access for hemodialysis.

FIG. 5. Illustration of drug eluted vascular graft used for vascular reconstruction in peripheral vascular disease.

FIG. 6. Illustration of drug eluted vascular graft used for coronary artery bypass grafting.

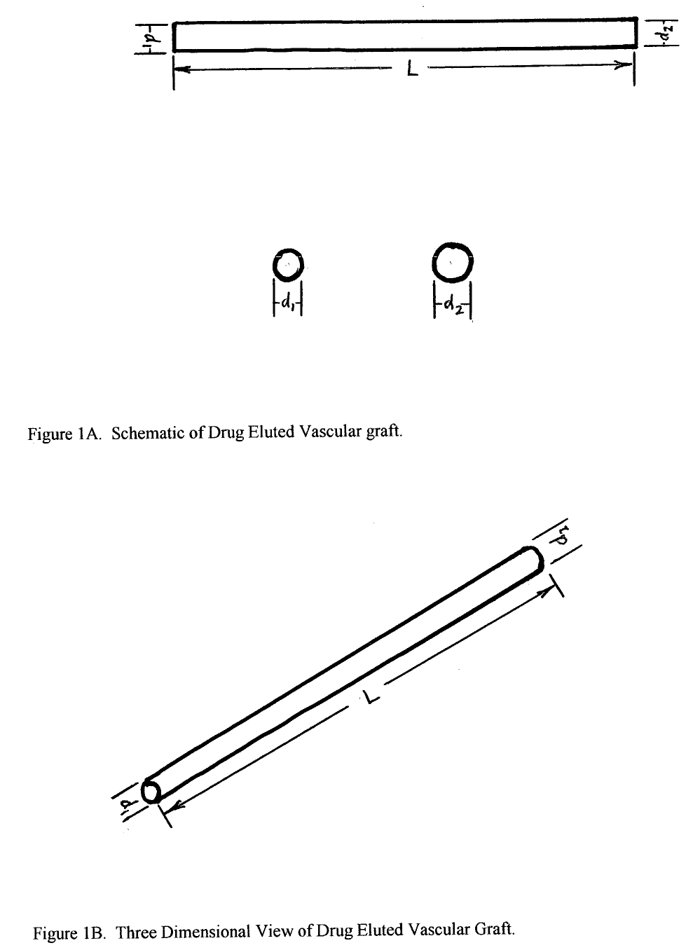

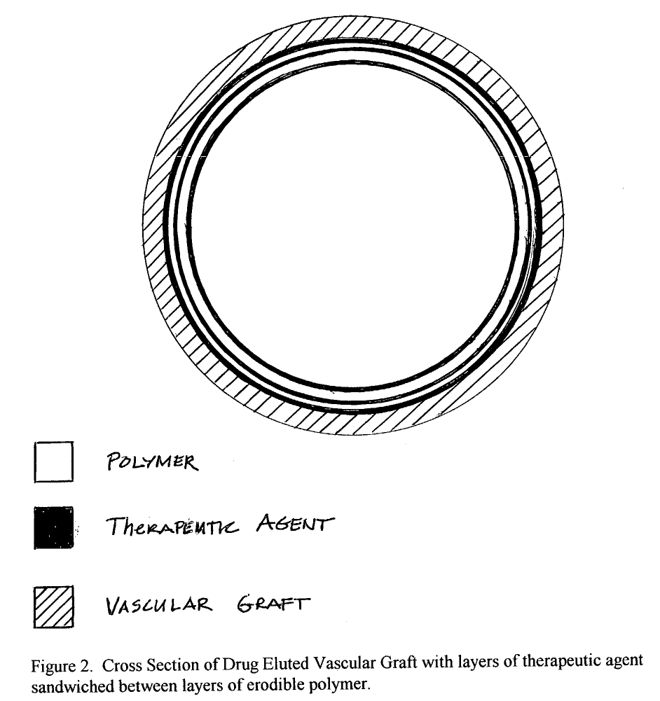

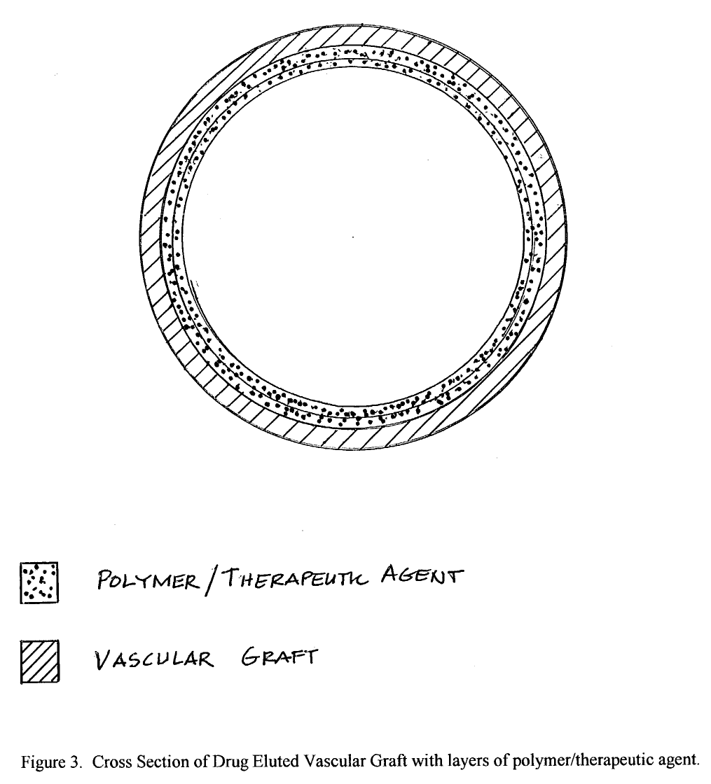

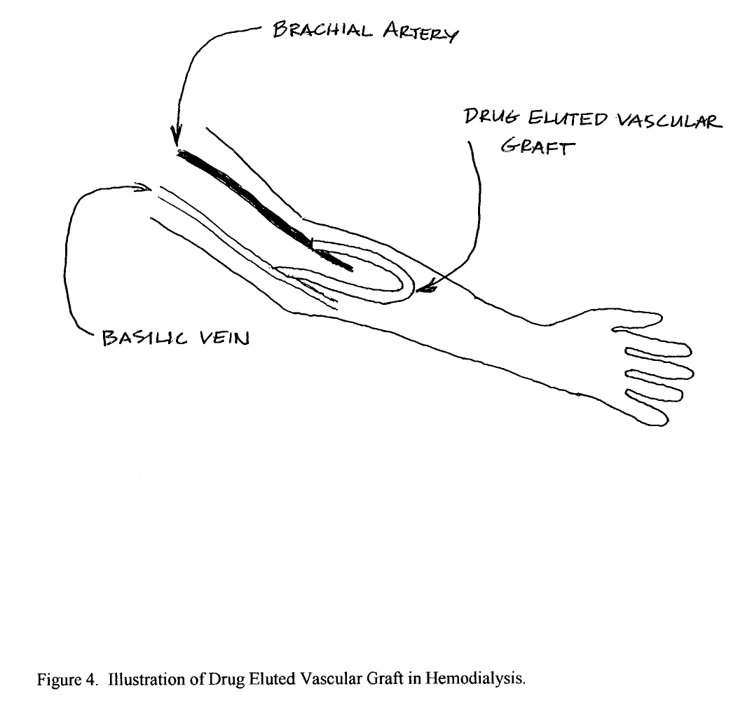

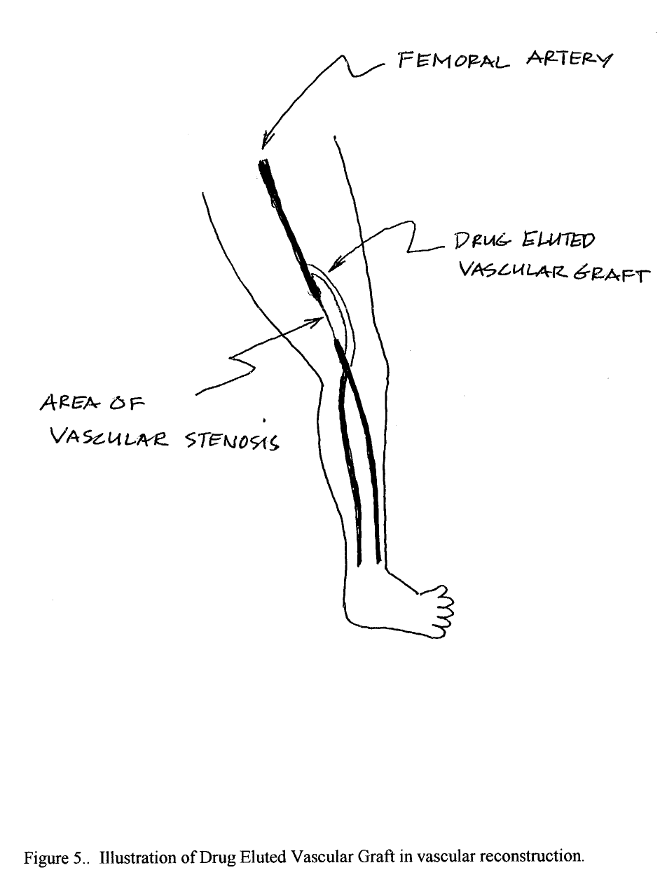

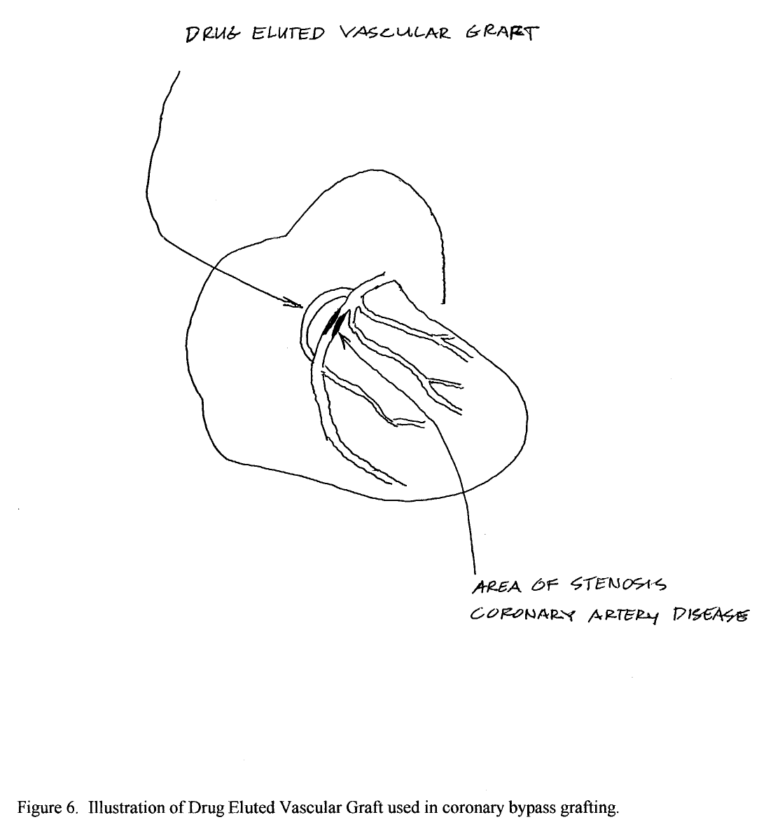

What is claimed:

1. A drug eluting vascular graft for the controlled and sustained delivery of at least one therapeutic agent within the internal lumen of the vascular graft.

2. The drug eluting vascular graft of claim 1, wherein said vascular graft is composed of at least one or more drug Eluted layers within the internal lumen of said graft.

3. The drug eluting vascular graft of claim 1, wherein drug an eluted layer(s) is composed of at least one bioerodible, biocompatible, pharmaceutically acceptable polymer.

4. The drug eluting vascular graft of claim 1, wherein microcapsules of one or more therapeutic agents are interspersed within said polymers.

5 The drug eluting vascular graft of claim 1, wherein said vascular graft may be comprised of expanded polytetrafluroethylene (ePTFE), polyvinylchloride polypropylene, florinated ethylene propylene, polyetherurethaneura or other biocompatible plastics.

6 The drug eluting vascular graft of claim 1, wherein said vascular graft may be of various diameters and lengths.

7 The drug eluting vascular graft of claim 1, wherein at least one polymer is a polyester.

8. The drug eluting vascular graft of claim 1, wherein at least polymer is of a pharmaceutically acceptable, biocompatible, bioerodible polymer comprising of mers I and II according to the following formula: ##STR1## wherein R.sub.1 is a member selected from the group consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with a member selected form the group consisting of alkyl of 1 to 7 carbons, an alkoxy of 1 to 7 carbons, and alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, an alkoxy of 1 to 7 carbons, an alklene of 1 to 10 carbons, an alkenyl of 2 to 7 carbons; and wherein a is 0 to 1; b is 2 to 6; m is greater than 10; n is greater than 10; and at least one R.sub.1, a, and b in mer I is different that R.sub.1, a, and b in mer II; a drug present in the matrix; and (c) wherein the device when in operation bioerodes and releases drug at a rate selected from (1) a zero order rate, (2) a continuous rate, and (3) a variable rate, which rate is produced by preselecting the copolymer, the drug, and the geometric device to give the desired result.

9. The drug eluting vascular graft of claim 1, wherein at least one polymer is of a hydrophobic, bioerodible, drug release rate controlling material, which material is comprising mers I, II, and III according to the following formula: ##STR2## wherein R.sub.1 is a member selected from the group consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with a member selected from the group consisting of alkyl of 1 to 7 carbons, alkoxy of 1 to 7 carbons, and alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, an alkoxy of 1 to 7 carbons, and alkylene of 1 to 10 carbons, an alkenyl of 2 to 7 carbons; and wherein a is 0 to 1; b is 2 to 6; m is greater than 10; n is greater than 10; p is greater than 10; and a least one of R.sub.1, a, and b in mers I, II, and III is different than R.sub.1, a, and b in mers I, II, and III; (b) a drug present in the matrix; and (c) wherein the device when in operation bioerodes and releases drug at a controlled rate selected from (1) a zero order rate, (2) a continuous rate, and (3) a variable rate, which different rate is achieved by a preselected the terpolymer, the drug and the geometric shape forming the device.

10. The drug eluting vascular graft of claim 1, wherein the graft comprises: (a) a matrix shaped, sized and adapted for placement in a human environment for administering drug thereto; (b) a multiplicity of microcapsules housed in the matrix with the microcapsules having a wall formed of a drug release rate controlling material, (c) a drug selected from the group consisting of locally and systemically acting therapeutic agents in the microcapsules; said matrix formed of a bioerodible drug release rate controlling pharmaceutically acceptable material, which material comprises a polymer of The formula ##STR3## wherein R.sub.1 is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkoxy of 1 to 7 carbons, an alkylene of 1 to 10 carbons, and an alkenyl of 2 to 7 carbons; cycloalkenylene of 4 to 7 carbons cycloalkenylene of 4 to 7 carbons substituted with an alkyl of 1 to 7 carbons, and alkoxy of 1 to 7 carbons, and alkylene of 1 to 10 carbons, and alkenyl of 2 to 7 carbons; arylene; and arylene substituted with an alkyl of 1 to 7 carbons, and alkoxy of 1 to 7 carbons, and alkenyl of 2 to 7 carbons; R.sub.2 and R.sub.3 are selected from the group consisting alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; aralkenyleneoxy of 8 to 12 carbons; oxa; OR.sub.1 O with R.sub.1 as defined above; a heterocyclic ring of 5 to 8 carbon and oxygen atoms formed when R.sub.2 and R.sub.3 are taken together; a heterocyclic ring of 5 to 8 carbon and oxygen atoms substituted with an alkyl of 1 to 7 carbons, alkoxy of 1 to 7 carbons and alkenyl of 2 to 7 carbons formed when R.sub.2 and R.sub.3 are taken together; a fused polycyclic ring of 8 to 12 carbon and oxygen atoms formed when R.sub.2 and R.sub.3 are taken together; a fused polycyclic ring of 8 to 12 carbons and oxygen atoms substituted with an alkyl of 1 to 7 carbons; an alkoxy of 1 to 7 carbons and an alkenyl of 2 to 7 carbons; and wherein at least one of said R.sub.2 and R.sub.3 is a member selected form the group consisting of alkoxy, alkenyloxy and OR.sub.1 O; R.sub.2 and R.sub.3 when taken together are a member selected from the group of heterocyclic and fused polycyclic rings having at least one oxygen atom in the ring; and wherein n is greater than 10; (d) wherein, when the device is in operation, the matrix and the bioerode at a controlled and continuous rate over a prolonged period of time, thereby administering a therapeutically effective amount of drug to the patient at a controlled and continuous rate over a prolonged period of time.

11. The drug eluting vascular graft of claim 1, wherein vascular graft comprises: (a) a matrix shaped, sized, and adapted for administering therapeutic agent to an animal, said matrix comprising multi layers formed of different bioerodible drug release rate controlling pharmaceutically acceptable materials, selected from materials which comprise a polymer of the formula: ##STR4## wherein R.sub.1 is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkylene of 1 to 10 carbons, and an alkenyl 2 to 7 carbons; cycloalkenylene of 4 to 7 carbons; cycloalkenylene of 4 to 7 carbons substituted with an alkyl of 1 to 7 carbons, an alkoxy of 1 to 7 carbons, an alkylene of 1 to 10 carbons, and an alkenyl of 2 to 7 carbons; arylene; and arylene substituted with an alkyl of 1 to 7 carbons, and alkoxy of 1 to 7 carbons, and an alkenyl of 2 to 7 carbons; R.sub.2 and R.sub.3 are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; oxa; OR.sub.1 O with R.sub.1 as defined above; a heterocyclic ring of 5 to 8 carbon and oxygen atoms formed when R.sub.2 and R.sub.3 are taken together; a heterocyclic ring of 5 to 8 carbon and oxygen atoms substituted with an alkyl of 1 to 7 carbons; an alkoxy of 1 to 7 carbons and an alkenyl of 2 to 7 carbons formed when R.sub.2 and R.sub.3 are taken together; a fused polycyclic ring of 8 to 12 carbons and oxygen atoms formed when R.sub.2 and R.sub.3 are taken together; a fused polycyclic ring of 8 to 12 carbon and oxygen atoms substituted with an alkyl of 1 to 7 carbons, and alkoxy of 1 to 7 carbon and an alkenyl of 2 to 7 carbons; and wherein at least one of said R.sub.2 and R.sub.3 is a member selected from the group consisting of alkoxy, alkenyloxy and OR.sub.1 O; R.sub.1 and R.sub.3 when taken together are a member selected from the group of heterocyclic and fused polycyclic rings having at least one oxygen atom in the ring; and wherein n is greater than 10; (b) a therapeutic agent selected from the group consisting of locally and systemically acting pharmaceutically acceptable drugs present in the layers; and, (c) wherein, when the vascular graft is in operation, the layers bioerode at a controlled and continuous rate over a prolonged period of time, thereby administering a therapeutically effective amount of drug to the animal at a controlled and continuous rate over a prolonged period of time.

12. A drug eluted vascular graft in claim 1, wherein the vascular graft comprises. (a) a matrix shaped, sized and adapted for administering therapeutic agent to an animal; (b) a plurality of discrete, closed cells in the matrix, said cells having a wall formed and defined by the matrix; said matrix formed of a bioerodible drug release rate controlling pharmaceutically acceptable material, which comprises a polymer of the formula: ##STR5## wherein R.sub.1 is a member selected from the group of divalent, trivalent and tetravalent radicals consisting of alkylene of 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, and alkoxy of 1 to 7 carbons, an alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, and alkoxy of 1 to 7 carbons, an alkylene of 1 to 10 carbons, and alkenyl of 2 to 7 carbons; arylene; and arylene substituted with an alkyl of 1 to 7 carbons, and alkoxy of 1 to 7 carbons, and an alkenyl of 2 to 7 carbons; R.sub.2 and R.sub.3 are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; aralkyleneoxy of 8 to 12 carbons; aralkenyleneoxy of 8 to 12 carbons; oxa; OR.sub.1 O with R.sub.1 as defined above; a heterocyclic ring of 5 to 8 carbon and oxygen atoms formed when R.sub.2 and R.sub.3 are taken together; a heterocyclic ring of 5 to 8 carbon and oxygen atoms substituted with an alkyl of 1 to 7 carbons, an alkoxy of 1 to 7 carbons and an alkenyl of 2 to 7 carbons formed when R.sub.2 and R.sub.3 are taken together; a fused polycyclic ring of 8 to 12 carbon and oxygen atoms formed when R.sub.2 and R.sub.3 are taken together; a fused polycyclic ring of 8 to 12 carbons and oxygen atoms substituted with an alkyl of 1 to 7 carbons, and alkoxy of 1 to 7 carbons and an alkenyl of 2 to 7 carbons; and wherein at least one of said R.sub.2 and R.sub.3 is a member selected form the group consisting of alkoxy, alkenyloxy and OR.sub.1 O; R.sub.2 and R.sub.3 when taken together are a member selected from the group of heterocyclic and fused polycyclic rings having at least one oxygen atom in the ring; an wherein n is greater than 10; (d) a therapeutic agent selected from the group consisting of locally and systemically acting pharmaceutically acceptable drugs present in the cells, said drug dissolved in a pharmaceutically acceptable carrier that is a solvent for the drug and a nonsolvent for the polymer; and, (e) wherein, when in operation, the vascular graft bioerodes at a controlled and continuous rate over a prolonged period of time, thereby administering a therapeutically effective amount of drug in the carrier to the animal at a controlled and continuous rate over a prolonged period of time.

13. The drug eluted vascular graft of claim 1, where in the vascular graft comprises: (a) a matrix shaped, sized and adapted for administering drug to an animal, which matrix is a multilayered with the layer formed oaf a bioerodible drug released rate controlling pharmaceutically acceptable material, said material selected form and comprising a polymer fo the formula: ##STR6## wherein R.sub.1 is a member selected from the group of divalent, trivalent, and tetravalent radicals consisting of alkylene 1 to 10 carbons; alkenylene of 2 to 10 carbons; alkyleneoxy of 2 to 6 carbons; cycloalkylene of 3 to 7 carbons; cycloalkylene of 3 to 7 carbons substituted with an alkyl of 1 to 7 carbons, alkoxy of 1 to 7 carbons, and alkylene of 1 to 10 carbons, and an alkenyl of 2 to 7 carbons; cycloalkenylene of 4 to 7 carbons; cycloalkenylene of 4 to 7 carbons substituted with an alkyl of 1 to 7 carbons, and alkoxy of 1 to 7 carbons, an alkylene of 1 to 10 carbons, and alkenyl of 2 to 7 carbons; arylene; and arylene substituted with an alkyl of 1 to 7 carbons, an alkoxy of 1 to 7 carbons, and an alkenyl of 2 to 7 carbons; R.sub.2 and R.sub.3 are selected from the group consisting of alkyl of 1 to 7 carbons; alkenyl of 2 to 7 carbons; alkoxy of 1 to 7 carbons; alkenyloxy of 2 to 7 carbons; alkylene of 2 to 6 carbons; alkenylene of 3 to 6 carbons; alkyleneoxy of 2 to 6 carbons; alkenyleneoxy of 3 to 6 carbons; aryloxy; arakyleneoxy of 8 to 12 carbons; aralkenyleneoxy of 8 to 12 carbons; oxa; OR.sub.1 O with R.sub.1 as defined above; a heterocyclic ring of 5 to 8 carbons and oxygen atoms formed when R.sub.2 and R.sub.3 are taken together; a heterocyclic ring of 5 to 8 carbon and oxygen atoms substituted with an alkyl of 1 to 7 carbons, an alkoxy of 1 to 7 carbons and an alkenyl of 2 to 7 carbons formed when R.sub.2 and R.sub.3 are taken together; a fused polycyclic ring of 8 to 12 carbon and oxygen atoms formed when R.sub.2 and R.sub.3 are taken together; a fused polycyclic ring of 8 to 12 carbon and oxygen atoms substituted with an alkyl of 1 to 7 carbons, and alkoxy of 1 to 7 carbons and an alkenyl of 2 to 7 carbons; and wherein at least one of said R.sub.2 and R.sub.3 is a member selected from the group consisting of alkoxy, alkenyloxy and OR.sub.1 O; R.sub.2 and R.sub.3 when taken together are a member selected from the group of heterocyclic and fused polycyclic rings having at least one oxygen atom in the ring; and wherein n is greater than 10; (b) a plurality of discrete, closed cells in at least one layer; (c) a drug selected from the group consisting of locally and systemically acting therapeutically acceptable drugs mixed with a pharmaceutically acceptable carrier housed in the cells; and (d) wherein, when in operation, the vascular graft bioerodes at a controlled and continuous rate over a prolonged period of time, thereby administering a therapeutically effective amount of drug mixed with the carrier to the animal at a controlled and continuous rate over a prolonged period of time.

14. The drug eluted vascular graft of claim 1, wherein said vascular graft may contain a erosion rate modifier.

15. The drug eluting vascular graft of claim 1, wherein said therapeutic agent is selected from a group consisting of:
a). Antimicrotuble agent such as paclictaxel, docetaxel.
b). Antiproliferative agent such as cyclophosphamide, actinomycin-D, cis-platinum, mitomycin, methotrexate, azithioprim.
c). Immunosupressive agent such as sirolimus, cyclosporine A, or steroid such as dexamethasone or methylprednisolone.
d). A glycoprotein IIb/IIIa receptor inhibitor such as abciximab, eptifibatide, tirofiban, sibrafiban, xemilofiban, orbofiban, roxifiban, lotrabian.
f). A platelet aggregation inhibitor such as clopidogrel or ticlopidine.
g). A nitric oxide donor such as nitroglycerin, isosorbide dinitrate, or ntiroprusside.
h). A calcium channel blocker (verapamil, diltiazem, nifedipine, etc.).
I). An antithrombogenic agent (heparin, low molecular weight heparin, hirudin).
J). An anti-sense nucleic acid.
k). A thiazolidinedione.
l). A HMG Co A reductase inhibitor such as pravastatin.
m). An angiotensin converting enzyme inhibitor.
n). A omega 3 fish oil.

16. The drug eluting vascular graft in claim one, wherein microcapsules of said therapeutic agents are dispersed within said polymers.

17. The drug eluting vascular graft of claim 1, wherein said microcapsules have a wall formed of a drug release rate controlled material so that said microcapsules are released in a controlled and continuous rate over a prolonged period of time.

18. A method for preventing or thrombosis of vascular grafts.

19. A method for preventing stenosis of vascular grafts.

20. An improved vascular graft used in vascular access for hemodialysis.

21. An improved vascular graft used in vascular reconstruction.

22. An improved vascular graft used in the treatment of coronary artery disease.
